(19) United States
(12) Patent Application Publication
(10) Pub. No.: US 2013/0280229 A1
(43) Pub. Date: Oct. 24, 2013

(54) Stabilisation of Proteins

(71) Applicant: Arecar limited, Cambridge (GB)

(72) Inventors: Jan JEZEK, Wellingborough (GB); Charles POTTER, Oxfordshire (GB)

(21) Appl. No.: 13/917,937

(22) Filed: Jun. 14, 2013

Related U.S. Application Data
(63) Continuation of application No. 13/851,328, filed on Mar. 27, 2013.
Application No. 13/851,328 is a continuation of application No. PCT/GB2009/050867, filed on Jul. 16, 2009.

(30) Foreign Application Priority Data
Jul. 16, 2008 (GB) 0813006.4

(51) Int. Cl. A61K 9/19; A61K 38/44

(52) U.S. Cl. CPC A61K 9/19; A61K 38/443; A61K 38/44
USPC 424/94.4

(57) ABSTRACT

A dry composition for use in therapy or diagnosis, obtainable by drying an aqueous composition comprising a protein and one or more displacement buffers, wherein the pH of the aqueous composition is such that the protein is stable, wherein the or each displacement buffer has a pKₐ that is at least 1 unit greater or less than the pH of the aqueous composition, and wherein the aqueous composition is substantially free of a conventional buffer having a pKₐ that is within one pH unit of the pH of the aqueous composition.

### CROSS-REFERENCE TO RELATED APPLICATIONS

This application is a continuation of U.S. patent application Ser. No. 12/851,328 filed on Aug. 5, 2010. which is a continuation of International Application No. PCT/GB2009/050867, winch designated the United States and was filed on Jul. 16, 2009, which claims priority under 35 U.S.C. §119 or 365 to United Kingdom Application No, 0813006.4, filed on Jul. 16, 2008. The entire teachings of the above applications are incorporated herein by reference.

### FIELD OF THE INVENTION

This invention relates to the stability of proteins, particularly the stability of proteins in solid state, e.g. in frozen condition or following partial or substantial removal of water such as by drying or freeze-drying.

### BACKGROUND OF THE INVENTION

Many proteins, e.g., enzymes, antibodies or therapeutic proteins are unstable and are susceptible to structural degradation and consequent loss of activity while stored. The processes involved in protein degradation can be divided into physical (i.e. processes affecting non-covalent interactions, such as loss of quaternary, tertiary or secondary structure, aggregation, surface adsorption) and chemical (i.e. processes involving a covalent change such as de-amidation, oxidation, disulphide scrambling etc.). The rates of the degradation processes are proportional to temperature. Proteins are consequently generally more stable at lower temperatures.

In general, proteins are more stable in the absence of water. Most commercial proteins are therefore formulated as lyophilised powders. A typical lyophilised commercial protein formulation always comprises a buffer, such as phosphate buffer, and one or more additives. The additives may include one or more of the following:
Bulking agents: typically sugars or sugar alcohols such as sorbitol, sucrose, lactose or mannitol.
Stabilisers: typically water replacement sugars such as trehalose or sucrose that can protect the protein structure during freeze-drying.
Tonicity, modifiers: typically inorganic sails and amino acids (commonly glycine or arginine). These excipients are used to adjust osmolality. Osmolarity (following re-constitution) is an important parameter of a protein formulation for therapeutic use.
Surfactants: may be effective to prevent adsorption of proteins onto solid surfaces, agitation-induced aggregation and damage during freeze-drying.

Some proteins are known to be formulated, mid stored in solutions. Historically, this reduces production cost considerably at the expense of low stability. Aqueous solutions of proteins are often formulated in early stage development of a protein product during which the stability demands are not as strict as those for the final product. Typically, aqueous protein solutions have to be stored strictly at 4° C. In most cases, structural degradation and loss of activity occur even at this temperature over a period of storage. The stability of aqueous formulations can be improved by freezing, but in some cases the freeze-thaw cycle can contribute to fee protein damage.

A typical aqueous protein solution is formulated in a conventional buffer, most commonly in phosphate butler pH 6.8-7.3, although other buffers such as acetate, TRIS, carbonate or citrate are also used at certain pH ranges. The formulations may also comprise one or more of tonicity modifiers, surfactants and stabilisers (see above).

As shown above, the nature of additives in commercial protein formulations can vary. However, the common feature of the commercial formulations of proteins both in dry and in aqueous format is the presence of a buffer. A buffer is required to maintain the pH of the formulation close to a given value. Many commercial proteins are formulated in phosphate buffer at pit close to 7. In some eases, other buffers and other pH can be used. Formulating at pH away from 7 is typically driven by the need to increase protein solubility, which, can be achieved at pH away from the isoelectric point of the protein.

The choice of buffer for formulating proteins follows the well-defined principles of acid-base equilibria and Brønsted-Lowry acid-base theory. Acid-base equilibria relate to the exchange of protons (H⁺; also referred to as hydrogen cations) between two chemical species. Whilst the species that is donating the proton, is referred to as the acid, the species that is accepting the proton is referred to as the base. So, in the following reversible process.

HA+B⁻⇄HB+A⁻

HA acts as acid and B⁻ acts as base. In the opposite direction HB acts as acid and A⁻ acts as base. The ability of a compound to donate or accept proton is expressed by the dissociation constant Kₐ which describes the equilibrium between the protonated and de-protonated form of a compound in aqueous solutions as follows:

HX+H₂O⇄H₂O⁺X⁻

K_{eq}=[H₃O⁺][X⁻][HX][H₂O]

Since the [H₂O]=Constant=55.5 M then:

Kₐ=K_{eq}[H₂O]=[H₃O⁺][X⁻]/[HX]

pKₐ=−log Kₐ

The pKₐ of any species is a function of temperature. Whilst in many eases, such as phosphate, citrate or acetate, the temperature dependence is small, some buffers (such as TRIS/HCl) exhibit change of pKₐ by as much as 0.03 unit per each ° C.

The degree of protonation of a chemical species with a given pKₐ value depends on the pH of the solution. If pH=pKₐ of the species in question then 50% of the species exists in the protonated form and the remaining 50% in de-protonated form. If pH is one unit lower than pKₐ then 90% of the species exists in the protonated Conn and 10% in the de-protonated form. Similarly, if pH is one unit higher than pKₐ then 10% of the species exists in the protonated form and 90% in the de-protonated form. Although the percentage of the protonated and de-protonated forms of a compound remains constant so long as the pH and the temperature are constant, this is a result of a dynamic equilibrium between the compound and surrounding molecules. In other words, there is a continuous dynamic exchange of protons between the acid-base species in a system while the overall protonation status of each species in the solution is maintained constant.

By donating or accepting protons in the pH range around its pKₐ the species acts as a buffer. The presence of a buffer thus results in small changes of pH if either an acid or a base is added to the solution. The species exerts maximum buffering capacity at pH=pKₐ, and its ability to maintain pit declines as the pH moves away from the pKₐ.

The choice of the appropriate buffer generally depends on the pH required. The generally accepted rule is that the pKₐ of the buffer must be no more than one unit away from the required pH to act as an efficient buffer. Preferably, however, the pKₐ is within 0.5 units away from the required pH in order to maximise the buffering capacity of the species. Most preferably the pKₐ of the buffer is equal to the required pH of the solution. In this case, the proportion of the protonated form and the deprotonated form of the buffer are 50% respectively and its buffering capacity is utilised to the full extent. Such solution is then most efficiently protected against changes of pH both in the acid and in the alkaline direction.

EP1314437 discloses an aqueous composition comprising an antibody and histidine, at pH 7.1. This composition is said to be stable with respect to aggregation. Subsequent description suggests that, for use, a buffer should be added.

WO2007/003936 describes an aqueous system comprising a protein and one or more stabilising agents. The stabilising agents have ionisable groups capable of exchanging protons with the protein and with the ionised product of water dissociation. The ionisable groups include first groups that are positively charged when protonated and uncharged when deprotonated, and second groups that are uncharged when protonated and negatively charged when deprotonated. The pH of the composition is within a range of protein stability that is at least 50% of the maximum stability of the protein with respect to the pH; alternatively, the pH of the composition is no more than 0.5 units more or less than the pH at which the composition has maximum stability with respect to pH. The disclosure is based on the observation that, while there is invariably a range of pH values for which a composition is relatively stable, the presence of certain excipients is desirable, It Is stated that a buffer may be added.

PCT/GB2008/000082 (unpublished at the first filing date of a patent application relating to the present invention) describes how to improve the stability of aqueous protein solutions. The disclosure is based on the discovery that buffers having a pKₐ at or near the pH of the solution are undesirable, when considering the protein's stability with respect to pH. Rather, the key to the present invention is choice of the appropriate pH while minimizing the protein's ability to exchange hydrogen cations. In particular, an aqueous system is disclosed, which comprises a protein and one or more additives, characterised in that
(i) the system is substantially free of a conventional buffer, i.e a compound with pKₐ within 1 unit of the pH of the composition at the intended temperature range of storage of the composition;
(ii) the pH of the composition is set to a value at which the composition has maximum measurable stability with respect to pH;
(iii) the one or more additives are capable of exchanging protons with the protein and have pKₐ values at least 1 unit more or less than the pH of the composition at the intended temperature range of storage of the composition.

By keeping a protein al a suitable pH, at or near a value at which the measurable stability is maximal, in the absence of a conventional butter, the storage stability of the protein can be increased substantially. Storage stability can generally be enhanced further, possibly substantially, by use of additives having pKₐ between 1 to 5 pH units, preferably between 1 to 3 pH units, most preferably between 1.5 to 2.5 pH units of the nil of the aqueous composition at the intended temperature range of storage of the composition. The presence of these additives also improves the pH stability of the formulation and is generally preferred. Within the context of the present invention such additives are referred to as “displacement buffers”. Based on the principles of the Brønsted-to wry acid-base theory the displacement buffers are further characterised by being either more than 90% de-protonated or more than 90% protonated at the pH of the aqueous composition. In contrast, the conventional buffers are both less, than 90% de-protonated and less than 90% protonated, typically around 50% de-protonated and 50% protonated, at the pH of the aqueous composition.

### SUMMARY OF THE INVENTION

The present invention is based on the realisation that the stability imparted to aqueous formulations by the technology described in PCT/GB2008/00082 can be utilised in dry formulations intended primarily for therapeutic or diagnostic use. and are suitable for delivering proteins to the human or animal body. For this purpose, the dry composition may be further formulated with one or more additives selected from excipients, bulking agents, stabilisers, disintegrants, binder and other therapeutic or diagnostic agents. The dry composition may be shaped into a debited form, e.g. particles or powders, needles etc.

A dry composition of the invention is obtainable by drying an aqueous composition comprising a protein and one or more displacement buffers, wherein the pH of the aqueous composition is that at which the protein is stable, wherein the or each displacement buffer has a pKₐ that is at least 1 unit greater or less than the pH of the aqueous composition, and wherein the aqueous composition is substantially free of a conventional buffer having a pKₐ that is within one pH unit of the pH of the aqueous composition. The pH of the aqueous composition is preferably that at which the loss of the function and/or native structure of the protein in the dry state obtainable from such composition is minimal or at least near the minimal level under defined conditions, such as storage at 4° C. or storage at 25° C. or storage at elevated temperatures,

According to one aspect of the invention, a dry composition for use in therapy or diagnosis, is obtainable by drying an aqueous composition comprising a protein and one or more displacement buffers, wherein the pH of the aqueous composition is such that the protein is stable, wherein the or each displacement boiler has a pKₐ that is at least 1 unit greater or less than the pH of the aqueous composition, and wherein the aqueous composition is substantially free of a conventional buffer having a pKₐ that is within one pH unit of the pH of the aqueous composition.

Alternatively, a composition of the invention may be defined as a dry composition for use in therapy or diagnosis, comprising a protein component which is a homogeneous mixture of a protein and one or more displacement buffers which, on reconstitution in water, gives an aqueous composition having a given pH, wherein the or each displacement buffer has a pKₐ that is at least 1 unit greater or less than the given pH, and wherein the mixture is substantially free of a conventional buffer having a pKₐ that is within one pH unit of the given pH.

According to a further aspect of the invention, a process for the preparation of a pharmaceutically acceptable composition comprises:
(i) mixing a protein with one or mote displacement buffers at a pH at which the protein is stable, wherein the or each displacement buffer has a pKₐ that is at least 1 unit greater or less than the pH of the aqueous composition, and wherein the aqueous composition is substantially free of a conventional buffer having a pKₐ that is within one pH unit of the pH of the aqueous composition,
(ii) drying the resultant aqueous composition; and
(iii) combining the resultant dry composition with one or more additives.

Drying provides a “protein component” in which, together with any residual water, the protein and displacement buffer(s) may exhibit homogeneity at the molecular level. This degree of homogeneity may not be conferred to further additives in the solid phase. The dry composition comprises, typically in. admixture with at least one other discrete component, a homogeneous mixture of the protein and displacement buffer(s). and this mixture gives an aqueous composition having said pH, on reconstitution. The pH of the aqueous composition following reconstitution is a key parameter of the dry compositions disclosed herein as it defines the protonation state of the additives used in the dry compositions. It is important to realise that whilst the concept of pH has a meaning only in aqueous solutions as it is directly re Sated to the concentration of protonated water, the protonation state of other non-volatile excipients, including conventional buffers and displacement buffers, will be maintained in dry compositions following partial or substantial removal of water. The key pH-related characteristics of conventional buffers or displacement buffers are thus directly transferable from aqueous solutions to the corresponding dry compositions after drying.

A dry composition of the invention may be administered to a subject, to treat a condition for which the protein is known to be useful. Alternatively, administration may follow reconstruction.

The presence of the displacement buffer(s) serves to enhance the stability of the protein, typically with respect to temperature, e.g. so that cold storage can be avoided and/or to provide longer shelf-life. The buffer(s) and other additives may also serve to enhance stability with respect to ionising radiation (used, for example, to sterilise the composition), further processing (e.g. drying of the aqueous composition, or milling, grinding, tableting or extrusion of the dry form), and after administration, where the protein may need to retain activity for a long period of time in the human body, depending on the release characteristics of the dosage form in which it is administered,

### DESCRIPTION OF THE INVENTION

As discussed in more detail below, a dry composition of the invention may be prepared from an aqueous formulation of the type described in PCT/GB2008/000082 by a variety of procedures. The composition will typically contain residual water, although the amount of water will be reduced, e.g. to less than 10%, preferably less than 5%, more preferably less than 2 or 3%, and often less than 5%, by weight of the dry composition. Such a dry composition may be described below as a “solid dose system”.

For the purposes of illustration, step (i) of the process of the invention will generally involve mixing the protein and one or more displacement buffers in water. However, the use of water is not essential it is contemplated that any suitable solvent, e.g. ethanol, whether or not in admixture with water, may be used.

The invention further encompasses methods of making the solid dose systems, Suitable drying procedures include spray-drying, freeze-drying, air-drying, vacuum-drying, fluidised-bed drying, co-precipitation and super-critical fluid evaporation.

Components of the aqueous composition to be dried are fully described in PCT/GB2008/000082. The content of that specification is incorporated herein by reference. That specification is included as an Appendix, and it is to be understood that each feature described there may be relevant also to the present invention.

The invention is applicable to proteins of any molecular weight. The term “protein” is used herein to encompass molecules or molecular complexes consisting of a single polypeptide, molecules or molecular complexes comprising two or more polypeptides and molecules or molecular complexes comprising one or more polypeptides together with one or more non-polypeptide moieties such as prosthetic groups, cofactors etc. In particular, the invention relates to molecules having one or more biological activities of interest, which activity or activities are critically dependent on retention of a particular or native three-dimensional structure in at least a critical portion of the molecule or molecular complex.

Particularly preferred proteins for use in the invention are protein or peptide hormones and growth factors (e.g. insulin, glucagon, human growth hormone, gonadotropin, human thyroid stimulation hormone, granulocyte colony stimulation factor), therapeutic enzymes (e.g. streptokinase, asparaginase, uricase), recombinant vaccines (e.g. hepatitis B vaccine, malaria vaccine, human papilloma vaccine, meningitis A vaccine, meningitis C vaccine, pertussis vaccine, polio vaccines), therapeutic antibodies (e.g. anti-epidermal growth factor receptor (EGFR) monoclonal antibody, anti-HER2 monoclonal antibody, anti-CD52 monoclonal antibody, anti-CD20 monoclonal antibody, interferons and other therapeutic proteins such as erythropoietin, darbepoietin alpha (stimulating erythrocyte production), blood coagulation factors, such as factor VIII and factor IX or protein C.

The solid dose system may comprise a variety of additional components. Depending on their properties, their intended contribution to the system or their use, they may be incorporated tit the solution to be dried or added to the dry system. Preferably, the components in the system are optimised to provide the best stability for the protein, initially in solution, and then additional components are added when the solution has been dried. Addition may comprise simple or intimate admixture, by any means known to those skilled in the art, e.g. dry mixing, or milling.

Additional components that may be used, typically added to the dry system, include other therapeutic agents as well as additives and excipients that will typically be chosen according to the intended use. These include bulking agents, binding agents, desiccants, controlled release agents and disintegrants. Examples are well known. Preferred excipient/bulking agents include sugars or polyalcohols such as sucrose, lactose, marmitol, polyethylene glycol, dextrans or sorbitol. Binding agents include substances such as sorbitol and polyvinyl pyrrolidone. Disintegrants are included to help a solid dose system disintegrate and may include celluloses or cros-povidome. If controlled release of the protein is desired then controlled release agents may be incorporated, e.g. polylactide-co-glycolide (PLG), poly (lactic-co-glycolic) acid (PLGA), polycaprolactone, polyanhydride or a polyorthoester. Ideally, all of the components used in the solid dose system are Generally Regarded As Safe (GRAS).

Suitable additives include protein-stabilising agents such as protease inhibitors, chelating agent, anti-oxidants, preservatives, sugars and detergents. Such components may be added in step (i) of the process set out above.

However, the inventors have recognized that it may be important, in order not to adversely affect the stability of the formulation, to add other components only after drying in step (iii) of the process as set out above.

These include additives selected from excipients, bulking agents, dessicants, disintegrants and binders. Any other protein, or another therapeutic or diagnostic agent may also be added at this stage. The mixture that is obtained may be uniform, but would typically comprise, as discrete components, the ‘protein component’ that is homogeneous and obtainable from aqueous solution, and the one or more additives.

Preferably the aqueous composition (which may be the composition to be dried or the dry composition after reconstitution) contains less than 1 mM of conventional buffer. The or each displacement buffer is preferably at a concentration of 1 mM to 1 M, preferably 2 to 200 mM, and most, preferably from 5 to 100 mM.

Specific examples of dry compositions of the invention include those obtainable by drying an aqueous composition having a pH of about 5, comprising glucose oxidase and at least one additive selected from the group consisting of TRIS and lactate; by drying an aqueous composition having a pH of about 6.7, comprising catalase and at least one additive selected from the group consisting of TRIS, lysine and lactate; by drying an aqueous composition having a pH of about 8.3, comprising uncase and at least one additive selected from the group consisting of TRIS, lysine and lactate; by drying an aqueous composition having a pH of about 5, comprising Hepatitis B antigen and at least one additive selected from the group consisting of TRIS, histidine and lactate; or by drying an aqueous composition having a pH of about 6, comprising human growth hormone and at least one additive selected from the group consisting of TRIS, cytosine, purine and lactate.

Further examples are given below.

The protein component and optionally also any additional component is capable of reconstitution in water, Reconstitution will result in an aqueous composition with a pH reflecting the protonation state of the excipients in the dry state. The protonation state of the excipients is a key parameter of the compositions according to the present invention and it is therefore important to ensure minimal influence of the protonation conditions (i.e. pH) by the additional components, even if they are added to the system in the dry state subsequent to drying the protein component. So, the presence of the additional components will typically not cause a change in pH of more than 1 unit, preferably more than 0.5 unit, most, preferably more than 0.2 unit, as measured in aqueous solution following reconstitution of the dry composition.

The invention also encompasses methods of delivering protein by providing a solid dose system as described herein and administering the system to a subject. Administration can be mucosal, oral topical subcutaneous, intradermal, transdermal, intramuscular, intravenous, intranasal, intraocular or pulmonary, e.g. by inhalation.

Shaped compositions of the invention include particles, spherical or otherwise, e.g. for pulmonary delivery or formulations into tablets, pellets, implants, needles and fibres. In one embodiment, the solid dose system is sized and shaped for penetration of the epidermis and is suitable for ballistic delivery. A suitable size is in the range of 1-100 urn in diameter which allows penetration and delivery into the epidermis.

In another embodiment, the solid dose system is shaped like a needle. The stabilised, solution is dried and the dry stabilised material is mixed with selected binders and disintegrants. This composition can then be formed into a needle shape directly by compression or by extruding a length of material that can then be cut into a short rod with a point on one end. The advantage of having a needle shaped final formulation is that this can be pushed directly into the skin (dermal subcutaneous and/or intramuscular delivery) with a simple mechanism. A suitable mechanism is described in US2006/0161111. Once in the skin the formulation dissolves, releasing the therapeutic or diagnostic agent. For this embodiment the final formulation must have sufficient physical strength that it can penetrate the tissue without a needle. Alternatively, rods of the final formulation can be cut and these can be inserted into the body using a needle and trocar system.

Alternative preferred embodiments of the delivery systems include uniform microspheres, preferably with a narrow size distribution. This is useful for pulmonary delivery and formulation into other dosage forms. It is also useful when control of the depth of penetration of the system is desirable. Such control may be useful, for example, for intradermal, intramuscular, subcutaneous or intravenous delivery or the targeting of vaccines to the basal layer of the epidermis, to bring antigen into proximity to the Langerhan's cells of the skin, to induce optimal immune responses.

Systems suitable for transmucosal delivery include mucoadhesive wafers, films or powders, lozenges for oral delivery, pessaries, and rings and other devices for vaginal or cervical delivery.

Compositions suitable for gastrointestinal administration include powders, tablets, capsules and pills for ingestion and suppositories for rectal administration.

Compositions suitable for subcutaneous administration include various implants. Preferred implants are macroscopic discoid, spherical or cylindrical shapes for ease of insertion and may be either fast or slow release. Since the entire implant is dissolved in the body fluids, removal of the implant is not necessary. Furthermore, the implants do not contain synthetic polymers and are biodegradable.

Compositions suitable for ocular administration include microsphere and macrosphere formulations and saline chops, creams and ointments containing these and round-ended shaped rods which fit comfortably in the lower conjunctival fornix beneath the lower eyelid.

Compositions suitable for administration by inhalation include powder forms of the delivers systems. Preferably the powders are of a particle size in the range 0.1 to 10 microns. More preferably, 0.5 to 5 microns, yet more preferably 1 to 4 microns, and most preferably 2.5-3 microns.

Stabilised compositions in a dry form may be reconstituted and injected into the body with a standard needle and syringe. In these eases, the solutions that have stability of the protein optimised and then dried may still require bulking agents in the dry state in order that it is obvious to the user that there is material to be reconstituted. For example, microgramme quantities of a stabilised protein in a vial may not be obvious to the naked eye and so bulking agents will make it obvious that the vial has or has not been used.

Preferred solid dose systems/powders also contain an effective amount of a physiologically acceptable agent that reduces hygroscopicity to prevent substantial clumping, for instance, a 50% molar ratio of potassium sulphate. Sodium sulphate and calcium lactate are the preferred salts with potassium sulphate being the most preferred.

Atomizers and vaporisers filled with the powders are also encompassed by the invention. There is a variety of devices suitable for use in delivery of powders by inhalation, including dry powder inhalers. Devices suitable for use herein include those described in WO94/13271, WO94/08552, WO93/09832 and U.S. Pat. No. 5,239,993.

The compositions will often be more stable in a solid form and therefore it is possible to combine dry stabilised formulations of different proteins into one final dosage form providing not just better stability for the proteins but also better convenience for the patient. The proteins may not be optimally combined for stability in an aqueous form or may require different buffers, but in a dry state they will not have the same potential for cross-reactivity. It will be understood that, where this specification makes reference to “a protein”, more than one protein is included.

The following Examples illustrate the invention. In addition, any example of an aqueous foundation given in PCT/GB2008/000082 may be dried and used as described above.

### Example 1

### Glucose Oxidase

Dry samples of glucose oxidase were prepared by drying an aqueous solution of the enzyme (350 μg/mL) in glass vials adjusted to a particular pH in the presence of excipients. All compositions contained 100 mM NaCl (prior to drying). Drying was achieved under a stream of nitrogen at 30° C. and subsequent incubation at atmospheric, pressure in the presence of a desiccant. The vials were sealed and subjected to treatment at 70° C. for 48 hours. Following the heat-treatment the samples were reconstituted and analysed for remaining enzyme activity using the following procedure: Water was added to the sample to achieve 350 μg mL-1 of glucose oxidase. 50 μL of the solution was added to 50 mL of deionised water. The following solutions were then added:
10 mL of reagent mix (5 parts of 0.1 M sodium phosphate, pH 6+4 parts 2% w/w starch +1 part of 1 mg/mL lactoperoxidase enzyme);
5 ml, of 100 mM potassium iodide and 5 mL of 20% w/w glucose solution,

These were mixed together quickly. Time 0 was counted from the addition of the glucose. Alter 5 min, 1 ml of 5 M hydrochloric acid was added to stop the reaction. The absorbance was then read at 630 nm using a Unicam UV-visible spectrophotometer (Type: Helios gamma). If the colour intensity was too great to allow an accurate reading, the sample was diluted with a defined volume of deionised water to bring the colour back on scale. The results were expressed as a percentage of activity with respect to control samples stored at 4° C.

In spite of the removal of water from the formulations, the stability of glucose oxidase in the dry form was found to be dependent of the pH of the aqueous mixture from which the composition was dried. The optimum stability of glucose oxidase was found to be around pH 6 to 6.5. It was shown that in the presence of conventional buffers applicable in this pH range, namely histidine (pKₐ 6.1) or maleate (pKₐ 6.1) the activity recovery was considerably smaller compared with compositions in the absence of conventional buffers and in the presence of displaced buffer(s), such as a combination of glutamate (pKₐ4.2) and purine (pKₐ 8.8) or aspartate (pKₐ4.1) and lysine (pKₐ 9.0).

TABLE 1
Activity recovery of dry glucose oxidase following
incubation at 70° C. for 48 hours. All compositions
contained 100 mM NaCl prior to drying.

| | |
|---|---|
| | Activity recovery |
| | following incubation |
| Composition (prior to drying) | at 60° C. for 48 hours |

| | | |
|---|---|---|
| Phosphate (20 mM) | pH 4.5 | 32% |
| Phosphate (20 mM) | pH 5.0 | 44% |
| Phosphate (20 mM) | pH 5.5 | 61% |
| Phosphate (20 mM) | pH 6.0 | 71% |
| Phosphate (20 mM) | pH 6.5 | 68% |
| Phosphate (20 mM) | pH 7.0 | 50% |
| Phosphate (20 mM) | pH 7.5 | 29% |
| Histidine (20 mM) | pH 5.0 | 22% |
| Histidine (20 mM) | pH 5.5 | 27% |
| Histidine (20 mM) | pH 6.0 | 36% |
| Histidine (20 mM) | pH 6.5 | 39% |
| Histidine (20 mM) | pH 7.0 | 28% |
| Maleate (20 mM) | pH 5.0 | 31% |
| Maleate (20 mM) | pH 5.5 | 36% |
| Maleate (20 mM) | pH 6.0 | 44% |
| Maleate (20 mM) | pH 6.5 | 43% |
| Maleate (20 mM) | pH 7.0 | 36% |
| Glutamate (20 mM) + Purine (20 mM) | pH 5.0 | 79% |
| Glutamate (20 mM) + Purine (20 mM) | pH 5.5 | 86% |
| Glutamate (20 mM) + Purine (20 mM) | pH 6.0 | 93% |
| Glutamate (20 mM) + Purine (20 mM) | pH 6.5 | 91% |
| Glutamate (20 mM) + Purine (20 mM) | pH 7.0 | 77% |
| Aspartate (20 mM) + Lysine (20 mM) | pH 5.0 | 73% |
| Aspartate (20 mM) + Lysine (20 mM) | pH 5.5 | 81% |
| Aspartate (20 mM) + Lysine (20 mM) | pH 6.0 | 89% |
| Aspartate (20 mM) + Lysine (20 mM) | pH 6.5 | 90% |
| Aspartate (20 mM) + Lysine (20 mM) | pH 7.0 | 77% |

### Example 2

### Catalase

Dry samples of catalase were prepared by drying an aqueous solution of the enzyme (1 mg/mL) in glass vials adjusted to a particular pH in the presence of excipients. Ail compositions contained 100 mM NaCl (prior to drying). Drying was achieved under a stream of nitrogen at 30° C. and subsequent incubation at atmospheric pressure in the presence of a desiccant. The vials were scaled and subjected to treatment at 65° C. for 48 hours. Following the heat-treatment the samples were reconstituted and analysed for remaining enzyme activity using the following procedure; 2 mL of hydrogen peroxide (30 mM in water.) was added to 18 mL of PBS in a 125 mL polypropylene pot. 100 μL of sample (containing 100 μg mL-1 catalase) was added and mixed. The resulting mixture was incubated at room temperature precisely for 30 min. in the meantime, the following reagents were mixed in a plastic cuvette for spectrophotometric measurements:
2.73 mL of citrate/phosphate buffer (0.1 M, pH 5.0)
100 μL of TMB (3 mg/mL, dissolved in DMSO)
100 μL of lactoperoxidase

Following the 30 min incubation period, 70 μL of the catalase containing mixture was added to the cuvette and absorbance was read in approximately 30 s. The results were expressed as percentage recovery, by reference to the absorbance stored at 4° C.

The stability of catalase in the dry form was found to be dependent of the pH of the aqueous mixture from which the composition was dried. The optimum stability was found to be around pH 7.5. It was shown that in the presence of conventional buffers applicable in this pH range, namely phosphate (pKₐ 7.1) and TRIS (pKₐ 8.1) activity recovery was considerably smaller compared with compositions in the absence of conventional buffers and in the presence of displaced buffer(s), such as histidine (pKₐ 1 6.1, pKₐ2 9.0) or combination of maleate (pKₐ 6.1) and lysine (pKₐ 9.0).

TABLE 2
Activity recovery of dry catalase following incubation
at 65° C. for 48 hours. All compositions contained
100 mM NaCl prior to drying.

| | Activity recovery following | |
|---|---|---|
| Composition (prior to drying) | incubation at 60° C. for 48 hours | |
| Phosphate (20 mM) | pH 6.0 | 22% |
| Phosphate (20 mM) | pH 6.5 | 27% |
| Phosphate (20 mM) | pH 7.0 | 31% |
| Phosphate (20 mM) | pH 7.5 | 32% |
| Phosphate (20 mM) | pH 8.0 | 24% |
| TRIS (20 mM) | pH 6.0 | 11% |
| TRIS (20 mM) | pH 6.5 | 14% |
| TRIS (20 mM) | pH 7.0 | 23% |
| TRIS (20 mM) | pH 7.5 | 21% |
| TRIS (20 mM) | pH 8.0 | 8% |
| Histidine (20 mM) | pH 6.0 | 49% |
| Histidine (20 mM) | pH 6.5 | 52% |
| Histidine (20 mM) | pH 7.0 | 67% |
| Histidine (20 mM) | pH 7.5 | 73% |
| Histidine (20 mM) | pH 8.0 | 61% |
| Maleate (20 mM) + Lysine (20 mM) | pH 6.0 | 48% |
| Maleate (20 mM) + Lysine (20 mM) | pH 6.5 | 59% |
| Maleate (20 mM) + Lysine (20 mM) | pH 7.0 | 66% |
| Maleate (20 mM) + Lysine (20 mM) | pH 7.5 | 76% |
| Maleate (20 mM) + Lysine (20 mM) | pH 8.0 | 58% |

The Appendix follows.

1. A dry composition for use in therapy, obtainable by drying an aqueous composition comprising a protein and one or more displacement buffers, wherein the pH of the aqueous composition is such that the protein is stable, wherein the or each displacement buffer has a pKₐ that is at least one unit greater or less than the pH of the aqueous composition, wherein the aqueous composition contains less than about 1 mM of a buffer having a pKₐ that is within one pH unit of the pH of the aqueous composition and wherein the dry composition is shaped in the form of a particle, needle, implant, pellet or tablet.

2. A dry composition for use in therapy according to claim 1, which is shaped for use in therapy and is in the form of a needle.

3. A dry composition for use in therapy according to claim 1, further comprising protein-stabilizing agents, such as protease inhibitors, chelating agents, anti-oxidants, preservatives, sugars or detergents.

4. A dry composition for use in therapy according, to claim 1, wherein the or each displacement buffer has a pKₐ that is at least 1.5 units greater or less than the pH of the aqueous composition.

5. A dry composition for use in therapy according to claim 1, wherein the or each displacement buffer is present in the aqueous composition at a concentration from about 1 mM to about 1 M.

6. A dry composition for use in therapy according to claim 5, wherein the or each displacement buffer is present in the aqueous composition at a concentration from about 2 mM to about 200 mM.

7. A dry composition for use in therapy according to claim 6, wherein the or each displacement buffer is present in the aqueous composition at a concentration from about 5 mM to about 100 mM.

8-15. (canceled)

16. A dry composition for use in therapy according to claim 1, which comprises a therapeutic agent in addition to the protein.

17. A dry composition for use in therapy according to claim 1, which additionally comprises one or more additives selected from excipients, bulking agents, desiccants, disintegrants and binders.

18. A dry composition for use in therapy or diagnosis, comprising a protein component which is a homogeneous mixture of a protein and one or more displacement buffers which, on reconstitution in water, gives an aqueous composition having a given pH, wherein the or each displacement buffer has a pKₐ that is at least one unit greater or less than the given pH, and wherein the mixture is substantially free of a buffer having a pKₐ that is within one pH unit of the given pH, and wherein the dry composition is shaped for use in therapy and is in the form of a particle, needle, implant, pellet or tablet.

19-34. (canceled)

35. A dry composition for use in therapy according to claim 1, which contains less than 5% by weight water.

36. Use of a composition, according to claim 1, for the manufacture of a medicament for use in therapy of a condition for which the protein is useful.

37. A process for the preparation of a pharmaceutically acceptable composition, comprising;
(i) mixing a protein with one or more displacement buffers at a pH at which the protein is stable, wherein the or each displacement buffer has a pKₐ that is at least one unit greater or less than the pH of the aqueous composition, and wherein the aqueous composition contains less than about 1 mM of a buffer having a pKₐ that is within one pH unit of the pH of the aqueous composition:
(ii) driving the resultant aqueous composition;
(iii) combining the resultant dry composition with one or more additives; and
(iv) shaping, the drying composition such that the pharmaceutically acceptable composition is in the form of a particle, needle, implant or tablet.

38-41. (canceled)
